(19) United States
(12) Patent Application Publication
(10) Pub. No.: US 2006/0292112 A1
(43) Pub. Date: Dec. 28, 2006

(54) Dendrimer-photosensitizer complexes for medical applications

(76) Inventors: Beate Roder, Falkensee (DE); Steffen Hackbarth, Berlin (DE); Gisela Wohlecke, Berlin (DE)

Correspondence Address: BOLESH J. SKUTNIK PhD, JD, 515 Shaker Road, East Longmeadow, MA, 01028, US

(21) Appl. No.: 11/514,662

(22) Filed: Sep. 1, 2006

Related U.S. Application Data
(63) Continuation-in-part of application No. 10/048,683, filed on Jan. 16, 2003, now abandoned.

(30) Foreign Application Priority Data
Aug. 2, 1999 (DE) DE19936997.6

(51) Int. Cl. A61K 31/787; A61K 31/409; A61K 31/555

(52) U.S. Cl. 424/78.3; 514/185; 514/410; 977/906

(57) ABSTRACT

A method for enhanced Photodynamic Therapy (PDT) treatments by applying dendrimer-photosensitizer complexes to bring multiple photosensitizer moieties to a treatment site is provided. The most benefit is derived for treatments using systemic introduction of the photosensitizer formulation. Photosensitizers are covalently coupled to the peripheral bonding places of dendrimers, using labile bonds which are breakable in preference over bonding within either photosensitizer or dendrimer, and the photosensitizers are separated from the complexes in one or more successive cycles. Tetrapyrroles are the photosensitizers employed. In one embodiment the complex is also bound to an antibody or antibody fragment, which aids in targeting the complex to a desired treatment site. After introduction into a patient, the photosensitizers are controllably released, at the treatment site, from the complexes by either light, chemical, or a combined light/chemical effect. Generally the photosensitizers develop their full photodynamic activity as free molecules after being released from the complex. More than one type of photosensitizer may be bound in the complexes. Release and/or activation may be done in a single step or with repeated steps.

### REFERENCE TO A RELATED CASE

This application is a continuation-in-part of co-pending U.S. patent application Ser. No. 10/048,683 filed on Jan. 16, 2003 by Beate Röder, Steffen Hackbarth, Gisela Wöhlecke, inventors, entitled “DENDRIMER-PHOTOSENSITIZER COMPLEXES FOR MEDICAL APPLICATIONS”, and incorporated by reference herein.

### BACKGROUND OF THE INVENTION

1. Field of the Invention

The present invention relates to the transport and release of photosensitizers in PhotoDynamic Therapy (PDT) treatments to provide more efficient, effective and safer use of photosensitizers in these treatments. In particular, dendrimer-photosensitizers complexes, having multiple photosensitizers attached, transport photosensitizers to treatment sites and release them on command.

2. Current State of the Art

Photodynamic Therapy (PDT) as an application of photomedicine provides treatment methods for skin diseases, such psoriasis, viral infections, such as herpes, and cancerous diseases, such as skin carcinoma, and lung or bladder carcinomas. For mediating photodynamic activity, photosensitizers (PS) are used as dyes that are excited by radiation to long lived triplet states. Photodynamic activity arises from the triplet state by formation of singlet oxygen and/or formation of radicals.

A major recurring problem is using PDT in medical treatments is how to obtain selective accumulation of the PS moieties into targeted tissue. Since actively selective accumulation is not yet known, the necessity for creating a modular transport system arises. This transport system has to be able to transport the active substance to the target tissue. One way to achieve this goal is to use antibodies or antibody fragments. To maintain the activity of the antibodies, however, only a small number of PS can be coupled directly to the antibody, or antibody fragment. To transfer an adequate amount of PS to treatment sites, it would be beneficial to have a vehicle/compound which can bond/complex with several PS molecules and can also be coupled with an antibody or antibody fragment.

### OBJECTIVES AND BRIEF SUMMARY OF THE INVENTION

It is an objective of the present invention to enhance PDT treatments by application of a molecular complex which has multiple photosensitizers bound in it.

It is another objective of the present invention to provide a method wherein tetrapyrroles and dendrimers are complexed to form multi functional photosensitizers for PDT treatments.

It is still another objective of the present invention to provide a method to selectively transport photosensitizers to a treatment site by having dendrimer-photosensitizer complexes also bound to an antibody fragment.

It is further objective of the present invention to provide means for photosensitizers to be inactive until separated from a dendrimer-photosensitizer complex.

Briefly stated, the present invention provides a method for enhanced Photodynamic Therapy treatments by applying dendrimer-photosensitizer complexes to bring multiple photosensitizer moieties to a treatment site. The most benefit is derived for treatments using systemic introduction of the photosensitizer formulation.

Photosensitizers are covalently coupled to the peripheral bonding places of dendrimers, using labile bonds which are breakable in preference over bonding within either photosensitizer or dendrimer, and the photosensitizers are separated from the complexes in one or more successive cycles. Tetrapyrroles are the photosensitizers employed. In one embodiment the complex is also bound to an antibody or antibody fragment, which aids in targeting the complex to a desired treatment site. After introduction into a patient, the photosensitizers are controllably released, at the treatment site, from the complexes by either light, chemical, or a combined light/chemical effect. Generally the photosensitizers develop their full photodynamic activity as free molecules after being released from the complex. More than one type of photosensitizer may be bound in the complexes. Release and/or activation may be done in a single step or with repeated steps.

The above, and other objects, features and advantages of the present invention will become apparent from the following description read in conjunction with the accompanying drawings.

### BRIEF DESCRIPTION OF FIGURES

FIG. 1 presents the absorption spectra of pheophorbide a (1), pheophorbide a-succinimide ester (2), a mixture of pheophorbide a and dendrimers (3) and the pheophorbide a-16 dendrimer complex (4) in ethanol.

FIG. 2 illustrates the fluorescence spectra of pheophorbide a (11), pheophorbide a-succinimide ester (12), a mixture of pheophorbide a and dendrimers (13 and the pheophorbide a-16 dendrimer complex (14) in ethanol. The fluorescence intensity of (4) is strongly decreased while the shape of the spectrum is nearly unchanged.

FIG. 3 shows that the signal of the singlet oxygen luminescence increases by light exposure as a result of the detachment of the pheophorbide a molecules from the multiplier dendrimer.

### DETAILED DESCRIPTION OF PREFERRED EMBODIMENTS

It is beneficial to be able to have multiple molecules of photosensitizers in a treatment site to provide, quick and enhanced PhotoDynamic Therapy (PDT) at the selected site.

According to the present invention, the task of providing more photosensitizers at treatment sites is solved by using tetrapyrroles which are bound to the peripheral groups of dendrimers in an as high as possible number.

By the action of natural or artificial light, as well as laser light, a part or all of the PS molecules are separated [split off] from the dendrimer an develop their photodynamic action by absorption of light then. This process may be accomplished in one step or it can be repeated sequentially several times to free/activate a number of PS moieties at the treatment site. The tetrapyrroles used in this invention are compounds from the class of porphyrins, benzoporphyrins, chlorines, bacteriochlorins, porphycenes, as well as phthalocyanines and naphthalocyanines.

Preferred tetrapyrroles are chlorophyll and its natural derivatives, especially pheophorbide and pheophorbide derivatives. Especially preferred tetrapyrroles are those with an amphiphilic character by substitutions and which are only conditionally water soluble.

The advantages of the present invention is in the possibilities to apply highest active natural and/or synthetic PS in a process in which the PS molecules can be transported in a high number directly to the targeted cells.

The method of the present invention is especially advantageous because the PS can not undergo interaction with the biomolecules and thus PS will not dissolve into the circulating blood. Furthermore it is advantageous that the PS are separable from the dendrimers by action of light without the use of additionally chemical agents. Nevertheless in another embodiment a separation by chemical activators such as changes of pH value are also possible.

Another advantage of the present invention is that the PS are essentially faster accumulated in the target cell than by using other methods.

An optimization and adaptation of the photo toxic activity of dendrimer-photosensitizer complexes according to the present invention in each actual task can be varied by using different tetrapyrroles and/or dendrimers.

The present invention is further illustrated by the following examples, but is not limited thereby.

### EXAMPLE

For demonstration of the invention, a third generation diaminobutane-polypropylene-imine (DAB) dendrimer is used, which has 16 potential binding sites in its side groups for bonding with a dye. Pheophorbide a (Pheo) is isolated from dried leafs of stinging nettle (urtica urens) and activated with N-hydroxy succinimide.

1. Preparation of Pheo 16 (Pheophorbide a-diaminobutane-polypropylene-imine Dendrimer 3.0 Complex)

To start 15 mg of the DAB dendrimer (pre-dissolved in 1 ml of methanol and 2 drops of triethylamine) are dissolved in 10 ml of dichloromethane and stirred continuously. Then, 155 mg (25 equivalents) of the Pheo-succinimide ester dissolved in 10 ml dichloromethane are added. The solution is stirred for 24 hours at room temperature in the dark. Afterwards, the solution is washed with distilled water (Milli Q) several times and is then dried. 50 ml of methanol are added to the powder product to dissolve the free Pheo-succinimide ester molecules which were not bound to the dendrimers. After 6 hours, the supernatant is poured off and the remaining powder was dried. This procedure is repeated three times. The final product is a crystalline black powder.

To confirm its purity, 2 mg of the powder are dialyzed in 5 ml dichloromethane against 50 ml dichloromethane for three days in the dark. Neither Pheo-succinimide ester nor Pheo were found outside of the dialysis bag.

The covalent coupling of the Pheo to the dendrimers was also proven by MALDI.

2. Properties of Pheo 16

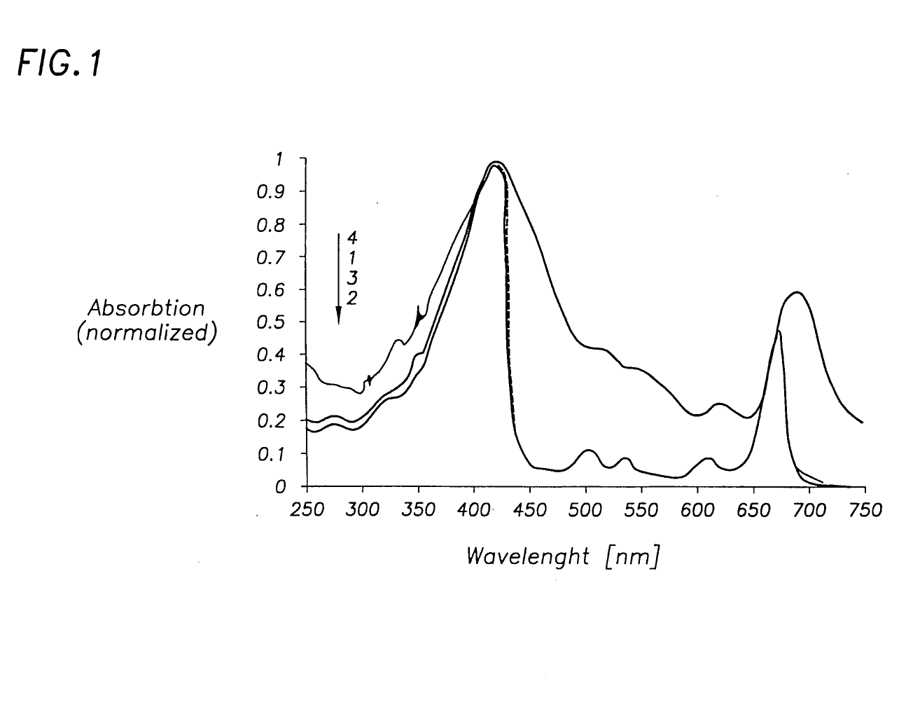

The absorption spectrum of Pheo-DAB in ethanol differs strongly from that of Pheo (see FIG. 1). The bandwidth of all absorption bands increase, the Q-bands are shifted bathochromically (5-14 nm), and the scattering ester.

The absorption spectrum of the mixture from Pheo and dendrimer is equal to the absorption spectrum of Pheo and the Pheo-succinimide ester.

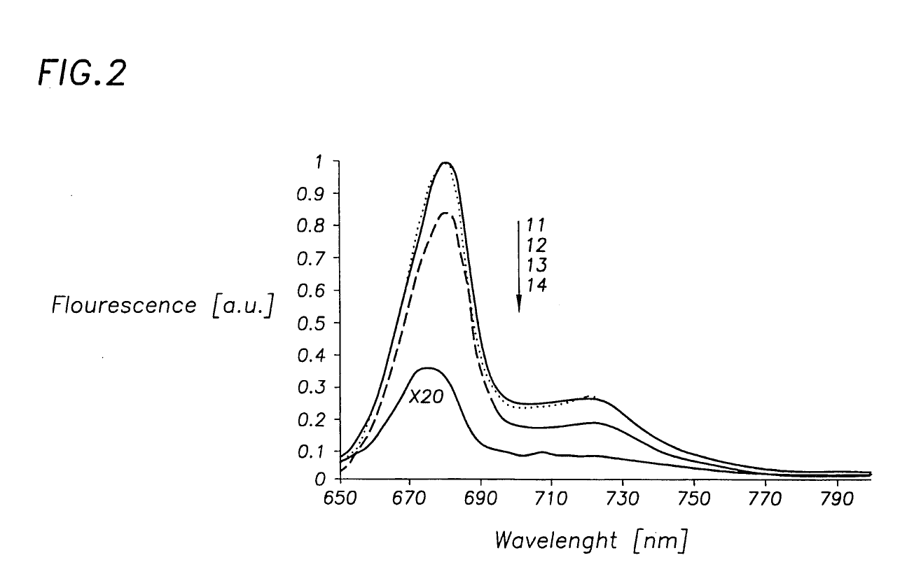

The fluorescence spectra of al samples show nearly the same shape. However, the fluorescence intensity of Pheo 16 in ethanol is 50 times smaller than the fluorescence intensity of Pheo in ethanol (see FIG. 2).

The fluorescence lifetime of Pheo in ethanol (5.7 ns) decreases when it becomes Pheo 16 and a double exponential decay is observed with 4.5 ng and 0.5 ns with a relation of amplitudes of 2 to 1, whereas the fluorescence lifetimes of the mixture or the Pheo-succinimide are similar to the Pheo lifetimes (see table 1).

The quantum yield of the photoinduced singlet oxygen of Pheo (0.52) decreases to 0.05 for Pheo 16 (see Table 1).

All of these findings indicated that the dye molecules are covalently bound to the dendrimer. The interaction between dye molecules is likely to be the reason for the strongly reduced fluorescence intensity and the generation of singlet oxygen.

TABLE 1
Fluorescence life time (τ_{Fl}) and singlet oxygen quantum yield
(Φ_{A}) of each component in ethanol.

| Sample | τ_{Fl} [ns] | | Φ_{A} |
|---|---|---|---|
| Pheophorbide a | 5.7 ± 0.2 | | 0.52 |
| Pheo-succinimide | 6.1 ± 0.3 | | 0.55 |
| ester | | | |
| Mixture Pheo + dendrimers | 5.9 ± 0.3 | | 0.48 |
| Pheo-16-dendrimer | 4.9 ± 0.3 | 0.5 ± 0.3 | 0.05 |
| complex | | | |

3. The Influence of Light

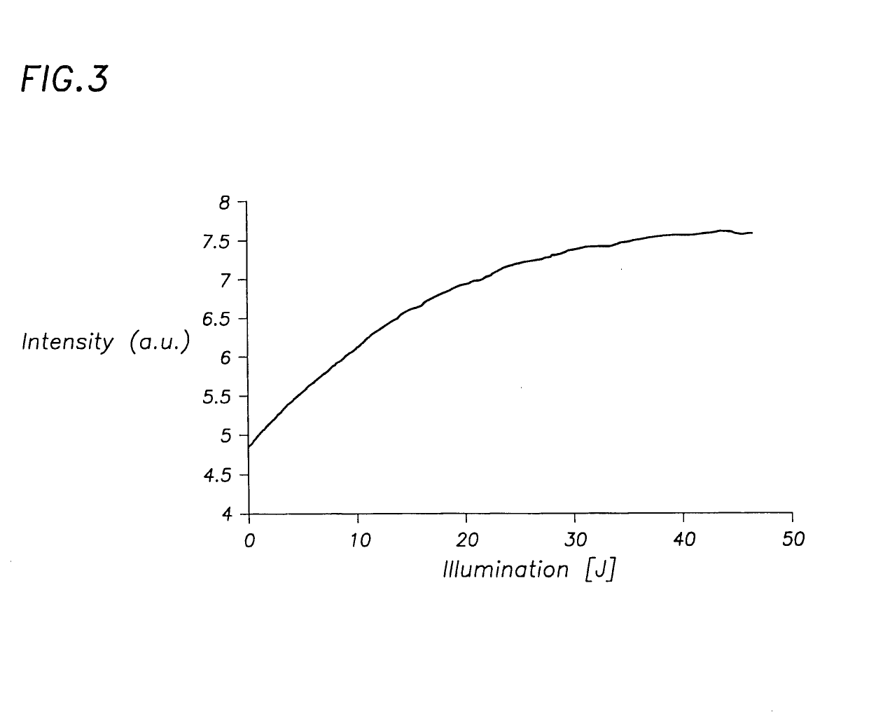

Surprisingly, the optical properties of Pheo 16 change dramatically by light exposure. They equal to the parameters of the free Pheo. The essential parameter changes are listed below:
The absorption spectrum of Pheo 16 changes.
The fluorescence intensity increases with exposure whereby the shape of the spectrum is maintained.
The singlet oxygen quantum yield of Pheo 16 increases after light exposure of a Pheo 16 sample (3 ml) with 40 J and 514 nm and reaches the value of 0.47 (see FIG. 3).
It was possible to confirm the release of Pheo and after 30 min light exposure of Pheo 16 with UV lamp (˜1 kJ) by MALDI).

The described effects show that the dye is split off from the dendrimer by light exposure and is photoactive as a monomer thereafter. However, this process occurs only in the presence of oxygen. Presumably, the primary generated singlet oxygen caused the separation of the bonds between the dye molecules and the dendrimer.

The results show that the described Pheo 16, on the one hand, is nearly photo inactive as long as the dye is covalently coupled to the dendrimer and, on the other hand, it is surprisingly possible to release the dye molecules by simple light exposure which occurs during the therapy of diagnostic session. Consequently, it is possible to call (request) for the photosensitizing activity of the dye at a distinct time. Surprisingly, the dye released by this way possesses the nearly identical properties as those of the free dissolved monomers. Thus, the described molecular complex (or similar complexes) could be used as an agent to administer multiple photosensitizers since it guarantees that the dye molecules bound to the dendrimer are not photoactive without light exposure and the photodynamic activity will be obtained momentarily upon exposure/activation.

Having described preferred embodiments of the invention with reference to the accompanying drawings, it is to be understood that the invention is not limited to the precise embodiments, and that various changes and modifications may be effected therein by skilled in the art without departing from the scope or spirit of the invention as defined in the appended claims.

What is claimed is:

1. A method of PhotoDynamic Therapy (PDT) at a preselected treatment site, wherein multiple photosensitizers (PS) are applied as dendrimer-photosensitizer complexes using tetrapyrroles as photosensitizers and dendrimers as a multi-functional substrate for said photosensitizers, comprising the steps of:
a) attaching a plurality of tetrapyrroles photosensitizers to a dendrimer substrate using a labile bond, wherein said labile bonds are breakable without disrupting bonding within said photosensitizers or within said dendrimer substrate;
b) introducing said dendrimer-photosensitizer complex to a patient, typically systemically;
c) allowing time for said dendrimer-photosensitizer complex to preferentially accumulate near a treatment site;
d) releasing, i.e. breaking one or more labile bonds between said photosensitizer and said dendrimer, said tetrapyrroles photosensitizers from said dendrimer substrate by irradiating with light selected from a group consisting of natural light and artificial light; and
e) irradiating with a second wavelength light specific to activate said freed photosensitizer to accomplish desired PDT of preselected treatment site.

2. The method according to claim 1, wherein said tetrapyrroles are selected from the group consisting of chlorophylls, pheophorbides, chlorines and bacteriochlorins, porphycenes, texaphyrines, sapphyrines, phthalocyanines, and naphthalocyanines.

3. The method according to claim 1, wherein said dendrimers are selected from the group consisting of starburst dendrimers, linear chains of dendrones and branched chains of dendrones.

4. The method according to claim 1, wherein said artificial light is from a laser.

5. The method according to claim 1, wherein said releasing step is performed by sequentially repeated light exposures.

6. The method according to claim 1, further comprising a step of releasing said tetrapyrroles from said dendrimer-photosensitizer complex by a chemical effect.

7. The method according to claim 6, wherein said chemical effect is selected from a group consisting of a change of pH and an enzymatic effect.

8. The method according to claim 1, further comprising a step of releasing said tetrapyrroles from said dendrimer-photosensitizer complex by a combination of light effect and chemical action.

9. The method according to claim 1, wherein said tetrapyrroles are activated by absorption of light, and where in further said light is selected from a group consisting of natural light and artificial light.

10. The method according to claim 9, wherein said artificial light is from a laser.

11. The method according to claim 1, wherein more than one type of dendrimer-photosensitizer complex is applied.

12. The method according to claim 1, wherein said dendrimer-photosensitizer complex are administered in more than one application.

13. The method according to claim 1, wherein said dendrimers-photosensitizer complexes are bound to antibodies or antibody fragments.

14. A multiple photosensitizer complex for PhotoDynamic Therapy comprising:
tetrapyrroles as photosensitizers;
wherein said tetrapyrroles are selected from a group consisting of chlorophyll, pheophorbide, porphyrins, chlorins and bacteriochlorins, porphycenes, phthalocyanines, and naphthalocyanines;
dendrimers as a multi-functional substrate for said photosensitizers; wherein said dendrimers are selected from a group of consisting of: starburst dendrimers, linear chains of dendrones and branched chains of dendrones; and
wherein said bonds between said photosensitizers and said dendrimer substrate are labile, i.e. breakable without disrupting bonding within said photosensitizers or within said dendrimer substrate.

15. A multiple photosensitizer complex according to claim 14, wherein said labile bonds are photosensitive.

16. A multiple photosensitizer complex according to claim 14, wherein said labile bonds are chemically activatible.

17. A multiple photosensitizer complex according to claim 14, wherein said labile bonds are activated by a combination of chemical and photon means.

18. A multiple photosensitizer complex according to claim 14, wherein said tetrapyrrole photosensitizers upon separation from said complex are activatible by absorption of light, specific to said photosensitizer.

19. A multiple photosensitizer complex according to claim 18, wherein said light is selected from a group consisting of natural light and artificial light.
